(19) United States
(12) Patent Application Publication
(10) Pub. No.: US 2021/0315870 A1
(43) Pub. Date: Oct. 14, 2021

(54) COMBINATORIAL TARGETED THERAPY METHODS

(71) Applicant: Postsurgical Therapeutics, Inc., Costa Mesa, CA (US)

(72) Inventor: Soonkap Hahn, Irvine, CA (US)

(21) Appl. No.: 17/227,992

(22) Filed: Apr. 12, 2021

Related U.S. Application Data
(60) Provisional application No. 63/008,554, filed on Apr. 10, 2020.

(51) Int. Cl. A61K 31/44; A61K 9/50; A61K 47/59

(52) U.S. Cl. CPC A61K 31/44; A61K 47/593; A61K 9/5084

(57) ABSTRACT

A combinatorial targeted therapy method for treating cancer, including metastatic cancer in a subject is provided, the method being designed to prevent unacceptable level of systemic toxicity in the subject and thus forced stoppage of the treatment, by performing initial molecular diagnostics to detect genomic alterations at each cancer site of the subject; for each cancer site, designing an initial combination targeted therapy by selecting a plurality of targeted drugs, based on the results of the initial molecular diagnostic at each cancer site; assigning each targeted drug to systemic or local delivery method, based on each targeted drug's properties; simultaneously treating all cancer sites according to the designed initial combination targeted therapy for each site, by delivering each targeted drug according to assigned delivery method to each targeted drug; and monitoring the progress of the cancer at each cancer site by performing follow-up molecular diagnostics at each cancer site.

### CROSS-REFERENCE TO RELATED APPLICATIONS

This application claims the benefit of U.S. Provisional Application No. 63/008,554, filed Apr. 10, 2020, which is hereby incorporated by reference, to the extent that it is not conflicting with the present application.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

### 1. Field of the Invention

The invention relates generally to methods of treating cancer and specifically to a combinatorial targeted therapy to treat cancer patients at early or metastatic stage and a method of reducing the systemic drug toxicity while controlling the resistance issue faced by the current targeted therapy.

### 2. Description of the Related Art

There were approximately 12.7 million new cases of cancer and 8 million people died in 2008 worldwide. This rate increased to 18.1 million new cases of cancer and 9.6 million deaths worldwide in 2018. This makes cancer the leading cause of death in the developed world and the second leading in the developing world. People with cancer have been increasing primarily due to longer lifespan and life style change in developing countries.

Cancer is a complex disease that results from uncontrolled division and growth of cells. The uncontrolled division and growth of cells are due to genomic alterations caused by environmental, lifestyle factors or inherited genetics. The environmental and lifestyle factors include pollution, tobacco use, obesity, infections and radiation.

Cancer is currently treated with various methods: 1) surgery, 2) chemotherapy, 3) targeted therapy and 4) radiation. Among them, both chemotherapy and targeted therapy are effective for treating cancer. However, chemotherapy can kill normal, healthy cells along with cancer cells thus causing some side effects. In contrast, targeted therapy (so called “precision medicine” or “personalized medicine”) interferes with molecules specific to cancer cells and kills only cancer cells. Although this selectivity improves overall survival rates and reduces side effects, the cancer cells eventually develop resistance to the therapy in virtually every patient.

There are two types of resistance: 1) preexisting resistance and 2) acquired resistance during treatment. Both types of resistance may involve a number of complex mechanisms. If a certain targeted drug blocks one mechanism, the cancer cells can mutate and activate other mechanisms to grow.

To reduce the limitations associated with the above resistance issue, various methods have been attempted. One of the methods is to combine and administer several targeted drugs simultaneously. This method blocks potential pathways that cancer cells may escape to, if the initial pathway is blocked. However, according to these methods, the targeted drugs are taken orally or intravenously. That is a serious problem. That is because each targeted drug asserts some systemic toxicity, causing side effects. Further, the toxicities asserted by several targeted drugs accumulate and cause some very serious systemic side effects. This systemic toxicity issue prohibits this combination targeted therapy method from being used more frequently by cancer patients and often needs to be stopped because unacceptable systemic toxicity levels are reached.

In addition, if the primary cancer is spread to other organ(s) as secondary cancer (metastatic cancer), there is no currently reliable treatment option to cure or even slow down the progression of the cancer. That is because the primary cancer or secondary cancer may have its own multiple heterogeneous resistance mechanisms, and thus, adequately treating both primary and secondary cancer sites may require a different combination targeted therapy for each cancer site. Currently, due to cumulative systemic side effects via oral or intravenous (IV) delivery, it is not possible to use a different combination targeted therapy for each cancer site. For example, if a cancer patient has 5 (five) cancer sites, each site requiring 2 (two) distinct targeted drugs, the systemic administration via oral or intravenous (IV) delivery of the cumulative 10 (ten) targeted drugs are likely to cause an unacceptable level of systemic toxicity in the cancer patient. Using the currently known targeted therapies, 10-30% of patients cannot continue treatment due to the cumulative toxicity.

Therefore, there is a need to solve the serious shortcomings of current cancer therapies described hereinabove.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description.

In an aspect, the combinatorial targeted therapy method disclosed herein may include five or six principal steps:
1) Performing initial molecular diagnostics to detect genomic alterations at each cancer site of a subject;
2) Designing and selecting an initial combination targeted therapy for each cancer site, based on the results of the initial molecular diagnostics;
3) Assigning of each targeted drug to systemic or local delivery, based on each targeted drug's properties;
4) Simultaneously delivering the initial combination targeted drugs using suitable delivery device;
5) Monitoring the progress of cancer at each cancer site by performing follow-up molecular diagnostics;
6) And, preferably, maintaining the initial combination targeted therapy or designing a follow-up combination targeted therapy, based on the follow-up molecular diagnostics for each cancer site.

The combinatorial targeted therapy method disclosed herein has the advantage of being useful for treating both post- and pre-surgery cancer patients at early or even metastatic stages. Specifically, the disclosed method has the benefit of reducing the limitations faced by the current cancer treatment methods when they attempt to control the resistance to drugs developed by the cancer cells. Further, the combinatorial targeted therapy method disclosed herein allows for selective and simultaneous combined targeted therapy at multiple cancer sites through both systemic and local means, while controlling cumulative systemic toxicity, which is a major shortcoming of the current treatment methods, as indicated hereinabove.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 illustrates a flowchart depicting the steps of the combinatorial targeted therapy method disclosed herein, according to an aspect.

FIG. 2A is a graph showing the in vitro release profile of sorafenib encapsulated in S20PA52 PLGA polymer.

FIG. 2B is a graph showing the in vitro release profile of sorafenib encapsulated in S20PA53 PLGA polymer.

FIG. 3 is a graph showing the in vitro release profiles of everolimus encapsulated in two different PLGA polymers, E10PA53 and E10PE77.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention.

It should be understood that, for clarity of the drawings and of the specification, some or all details about some structural components or steps that are known in the art are not shown or described if they are not necessary for the invention to be understood by one of ordinary skills in the art.

In order to address and solve the issues presented by current cancer therapies, the invention disclosed herein, in an aspect, teaches the use of local intratumoral injection (i.e., local delivery) along with oral or IV delivery to treat multiple cancer sites without causing serious systemic side effects, as it will be described in more detail hereinafter. The cumulative systemic side effects caused by oral or IV delivery can be reduced significantly by selectively delivering targeted drugs locally, which limits the systemic absorption of delivered targeted drugs, as it will be described in more detail hereinafter. The disclosed combinatorial targeted therapy addresses existing shortcomings of current therapies by maximizing efficacy, reducing the limitations associated with controlling cancer cells' resistance to targeted therapies and minimizing systemic side effects associated with existing targeted therapies.

The properties inherent to combinatorial targeted therapy provide this method with significant advantages over current therapies. The overall reduction of total targeted drugs delivered systemically reduces the associated systemic toxicity, allowing for a greater variety of targeted drugs to be delivered through local means. This advantage enables this process to counteract the limitations associated with the aforementioned resistance issues, while simultaneously minimizing the resulting toxicity. Due to the reduced systemic toxicity enabled by the disclosed method, i.e., the combinatorial targeted therapy, more options are provided for advanced stage cancer patients (metastatic cancer patients) as well as cancer patients with very little treatment options, such as pancreatic cancer patients.

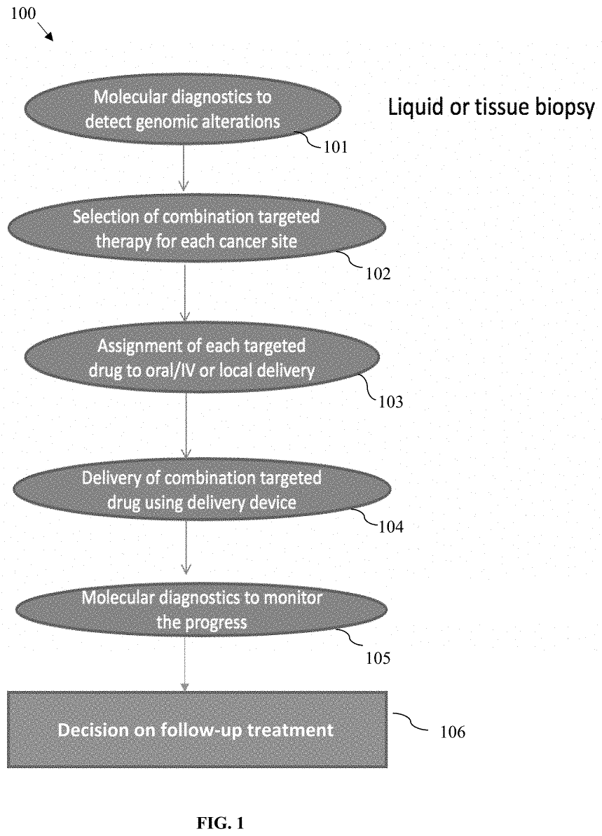

FIG. 1 illustrates a flowchart depicting the steps of the combinatorial targeted therapy method disclosed herein, according to an aspect. As shown, in an aspect, the combinatorial targeted therapy method 100 disclosed herein includes six principal steps: performing initial molecular diagnostics to detect genomic alterations (step 101), designing and selecting an initial combination targeted therapy for each cancer site based on the initial molecular diagnostics (step 102), assigning of each targeted drug to systemic or local delivery, based on each targeted drug's properties (step 103), delivering the initial combination targeted drugs using suitable delivery device or method (step 104), monitoring the progress of cancer at each cancer site by performing follow-up molecular diagnostics (step 105) and, preferably, maintaining the initial combination targeted therapy or designing a follow-up combination targeted therapy based on the follow-up molecular diagnostics (step 106).

Molecular diagnostics using liquid biopsy (i.e., blood) or tissue biopsy sample can detect genomic alterations (step 101) specific to each cancer patient and also specific to each cancer site, if the patient has more than one cancer site. Based on the information of these genomic alterations, oncologists can design and select an initial combination targeted therapy specific to the patient and, if applicable to that patient, to each cancer site (step 102).

Once the combination targeted therapy is determined, each drug in the initial targeted therapy can be assigned to either systemic (oral/IV) or local delivery (step 103) depending on its property. One drug property would be for example the known systemic toxicity level caused by the drug, so that drugs with high systemic toxicity would be assigned to local delivery instead of systemic delivery. Further, it is needed to deliver some drug(s) with low potency by oral delivery daily (i.e., requiring more than 500 mg oral delivery daily to be effective for treating cancer). That is because if a drug has low potency, it would have to be delivered in a very large amount locally, for a sustained, controlled release over a long period (e.g., over 1-6 months). However, it is difficult to deliver the very large amount locally. Thus, the systemic delivery is generally the better option for low potency drugs. From the above considerations, three rules may be used to determine proper delivery method assignments; low potency drugs requiring more than 500 mg oral delivery daily are to be delivered systemically, high potency drugs requiring less than 500 mg oral delivery daily are to be delivered locally and drugs causing frequent grade 3 or 4 systemic toxicity are to be delivered locally.

In addition, other considerations may need to be taken into account when assigning the drug to either oral/IV (i.e., systemic) or local delivery (step 103). For example, some types of cancer are difficult to reach locally. In that case, systemic delivery may need to be considered. However, if the drug is highly potent (i.e., requiring less than 50 mg oral delivery daily), endoscopic image-guided injection method, for example endoscopic ultrasound image-guided fine needle injection method, can be used for delivering a small amount of drug into tumor tissue. This injection method is complex and not ideal for frequent delivery. In addition, it is not easy to deliver a large amount of drug using this method.

Thus, in an example, if, let's say, two targeted drugs are needed for one cancer site that is accessible, both drugs will preferably be delivered locally. If however, one of the drug has low potency, that drug would be delivered systemically instead, by for example daily oral delivery. That is because, due to the typical high complexity of the local delivery procedure, local delivery may not routinely be used, e.g., daily.

In step 104, the targeted drug(s) are delivered using the suitable delivery device or method. Targeted drug encapsulated in biodegradable polymer such as polylactic glycolic acid (PLGA) can be delivered locally for sustained, controlled release over 1-6 months. For local delivery, if possible, tumor size or volume can be measured using imaging system such as ultrasound, MRI or other imaging system, before injection. The injected amount of targeted drug can be adjusted depending on the tumor size or volume. This adjustment is only possible through the implementation of local delivery and provides additional means of moderating system toxicity. This reliable means of drug adjustment is not possible with oral/IV delivery methods due to their indirect delivery method. Orally administered drugs must be absorbed through ingestion, and IV administered drugs must be carried through the bloodstream, which complicates drug adjustment determinations.

Targeted drug assigned for oral delivery can be taken as a tablet or capsule daily while that assigned for IV delivery is administered intravenously.

For local delivery, the delivery device can be for example a simple hand-held sprayer or image-guided delivery system. The simple hand-held sprayer is useful for treating post-surgery patients in the resected area after surgery. The image-guided delivery system is useful to deliver targeted drugs by intratumoral injection locally for pre-surgery patients. The image-guided delivery system has image-guided needle, syringe and imaging system. It can deliver targeted drugs in a powder form of microparticles or a solution form with suspended microparticles at multiple lesions. The imaging system includes ultrasound, CT, MM and other imaging system.

After the combinatorial targeted therapy is administered, the progress of cancer is preferably monitored by follow-up molecular diagnostics (step 105). Depending on the results of the follow-up molecular diagnostics, the initial combinatorial targeted therapy can be maintained or modified (step 106).

Again, cancer is a complex disease caused by uncontrolled division and growth of cells. This abnormal division and growth of cells is due to genomic alterations. These genomic alterations are caused by various mechanisms which are variable from one cancer type to another one as well as from one person to another person. For example, breast cancer may be caused by different genomic alterations compared to lung cancer. Even for the same breast cancer, there are three different subtypes in HER-positive, estrogen receptor positive and triple negative subtype.

Chemotherapy generally treats breast cancer patient with one of carboplatin, docetaxel, doxorubicin and paclitaxel by IV infusion regardless of their subtype. These drugs kill cancer cells as well as normal healthy cells causing side effects. In contrast, targeted therapy treats three subtypes of breast cancer patients differently based on their genomic alterations. For example, HER-2 positive patients have overexpressed HER-2 proteins. They are treated with lapatinib or trastuzumab which inhibits specifically the tyrosine kinase activity of HER-2. Since lapatinib and trastuzumab interfere only with HER-2, they are not effective for treating other subtype patients, estrogen receptor positive or triple negative patients. For estrogen receptor positive patients, a different combination targeted therapy of palbociclib and an aromatase inhibitor or fulvestrant can be used. Overall, this targeted therapy is more effective with less side effects compared to the above chemotherapy.

Another successful example is treating lung cancer with targeted therapy. About 85% of lung cancer is non-small cell lung cancer (NSCLC) subtype. The NSCLC patients can have multiple single nucleotide polymorphisms (SNPs) in epidermal growth factor receptor (EGFR). Patients with one or more of these mutations in EGFR respond well to EGFR tyrosine kinase inhibitors such as gefitinib and erlotinib, which block EGFR signaling. In patients without these mutations, EGFR is likely to function normally, so these drugs are unlikely effective. A similar approach can be taken for translocations in the ALK gene. Patients with ALK translocations respond well to treatment with crizotinib, a tyrosine kinase inhibitor which blocks the transmission of growth signals to the cell nucleus. As shown in these examples, the targeted therapy is more selective in treating cancer patients and has improved the overall survival rate significantly. With combinatorial targeted therapy, the use of these combination targeted therapies may be optimized to maximize efficacy, reduce the limitations associated with controlling cancer cells' resistance to targeted drugs and minimize systemic side effects. Through these optimizations, this method has the potential to provide more treatment options for both early and metastatic cancer patients.

Molecular diagnostics (step 101) is an essential method to detect genomic alterations. To detect genomic alterations, blood or tissue samples can be analyzed by various methods including next generation sequencing (NGS), fluorescence in situ hybridization (FISH) and other methods. Blood sample (liquid biopsy) has inherent advantage over tissue sample in terms of less invasiveness to collect and possibility of repeated samplings. Blood sample contains ct (circulating tumor) DNAs and circulating tumor cells (CTCs) shed from cancer cells into the blood stream. These are valuable to provide the information related to genomic alterations of each patient.

Once the genomic alterations are detected, oncologist can determine a matching targeted drug (step 102). For example, crizotinib is a right choice of targeted drug for a NSCLC patient with ALK translocations. A monotherapy of crizotinib is therapeutically effective initially for this patient. However, the patient eventually develops resistance to crizotinib by a mutation in ALK gene. By adding lorlatinib to crizotinib, the issues developed by the cancer cell's resistance are controlled and the patient can respond well. However, as stated hereinbefore, according to current methods, targeted drugs are taken either orally or intravenously. Again, this route of delivery has significant limitations on how many targeted drugs can be taken due to cumulative systemic side effects. Local delivery can reduce the systemic absorption of delivered targeted drugs significantly while maintaining their local level high.

The combinatorial targeted therapy method disclosed herein may also use oral/IV delivery as a systemic delivery method, along with local delivery. Again, as stated hereinbefore, in case a targeted drug has a low potency, it should be delivered orally. This dual mode of delivery may provide patients more treatment options and thus a higher likelihood of successful treatment of the cancer patients. The combinatorial targeted therapy method disclosed herein which combines oral/IV delivery with local delivery may provide a viable option to overcome the limitations associated with the developed resistances of cancer cells to targeted drugs and systemic toxicity issues described hereinbefore.

Again, as stated hereinbefore, currently, if the primary cancer is spread to other organ(s) as secondary cancer (metastatic cancer), there is no reliable treatment option to cure or even slow down the progression of the cancer. The primary cancer or secondary cancer may have its own multiple heterogeneous resistance mechanisms. Adequately treating both primary and secondary cancer sites may require a different combination targeted therapy for each cancer site. Currently, due to cumulative systemic side effects via oral or IV delivery, it is not possible to use current targeted therapy methods for each cancer site. The combinatorial targeted therapy method disclosed herein utilizes local intratumoral injection (i.e., local delivery) along with oral/IV administration to treat multiple cancer sites without causing serious systemic side effects. Local delivery of targeted drugs limits their systemic exposure while asserting their therapeutic effects to local cancer cells. Combining both local and oral/IV delivery may provide viable treatment option for both cancer patients at early stage and metastatic stage.

Once combination targeted therapy is determined (step 102), the oncologist needs to determine the route of delivery for each targeted drug, either oral/IV or local delivery. In this step, the most important parameter to consider is to maintain overall systemic toxicity below toxic level while maximizing efficacy. The three rules to apply in determining the delivery route are as follows; low potency drugs requiring more than 500 mg dose oral daily are delivered through oral/IV, high potency drugs requiring less than 500 mg dose oral daily are delivered through local means, and drugs causing frequent grade 3 or 4 systemic toxicity are delivered through local means.

The combinatorial targeted therapy method disclosed herein can be used by both post-surgery and pre-surgery cancer patients. When cancer is at an early stage (stage 1 or 2), the best treatment option is to remove tumor by surgery. Combinatorial targeted therapy can be used for prophylactic purpose to avoid the recurrence of cancer. In this case, combination targeted drugs encapsulated in PLGA microparticles can be sprayed by a simple hand-held sprayer. In addition, the combinatorial targeted therapy method disclosed herein may provide a viable treatment option for metastatic cancer patients. In this case, oncologist may design a combinatorial targeted drug regimen specific to each cancer site (i.e., primary cancer site as well as secondary cancer site(s)).

For local delivery, if possible, tumor size or volume can be measured using imaging system such as ultrasound, MRI or other imaging system before injection. The injection amount of targeted drug can be adjusted depending on the tumor size or volume. This adjustment capability is exclusive to local delivery methods, due to additional factors that may arise from systemic delivery methods that require the targeted drug to travel greater distances through the body before reaching the appropriate cancer site.

For local delivery, the combinatorial targeted therapy method disclosed herein uses an image-guided delivery system to deliver combination targeted drugs. Such a delivery system has an image-guided needle, syringe and imaging system. However, if a tumor is close to the skin or subcutaneous lesion or even palpable, the imaging system may not be needed. In case tumor lesion is not easily accessible, some additional instrument such as endoscopy may be needed to guide the needle. This delivery device can be linked with an imaging system such as ultrasound, MRI, CT or other imaging system. It can deliver combination targeted drugs as a powder or powder suspended in a solution at multiple lesions.

Each targeted drug can be encapsulated in a biodegradable polymer such as PLGA for sustained, controlled release over 1-6 months.

Targeted Drug Candidates

Targeted drugs block the growth and spread of cancer cells. Many targeted drugs have been approved by the FDA and others are currently being developed. The combinatorial targeted therapy method disclosed herein can use any of these drugs to design its combination targeted therapy. These targeted drugs include hormone therapies, signal transduction inhibitors, gene expression modulators, apoptosis inducers, angiogenesis inhibitors, immunotherapies, and toxin delivery molecules. The targeted drugs approved by the FDA are listed by cancer type in the following section:

Bladder cancer: atezolizumab, nivolumab, durvalumab, avelumab, pembrolizumab, erdafitinib

Brain cancer: bevacizumab, everolimus

Breast cancer: everolimus, tamoxifen, toremifene, trastuzumab, fulvestrant, anastrozole, exemestane, lapatinib, letrozole, pertuzumab, ado-trastuzumab emtansine, palbociclib, ribociclib, neratinib maleate, abemaciclib, olaparib, talazoparib tosylate, atezolizumab, alpelisib, fam-trastuzumab deruxtecan-nxki

Cervical cancer: bevacizumab, pembrolizumab

Colorectal cancer: cetuximab, panitumumab, bevacizumab, ziv-aflibercept, regorafenib, ramucirumab, nivolumab, ipilimumab

Dermatofibrosarcoma protuberans: Imatinib mesylate

Endocrine/neuroendocrine tumors: lanreotide acetate, avelumab, lutetium Lu 177-dotatate, iobenguane I 131

Endometrial cancer: pembrolizumab, lenvatinib mesylate

Esophageal cancer: trastuzumab, ramucirumab, pembrolizumab

Head and neck cancer: cetuximab, pembrolizumab, nivolumab

Gastrointestinal stromal tumor: imatinib mesylate, sunitinib, regorafenib

Giant cell tumor of the bone: denosumab

Kidney cancer: bevacizumab, sorafenib, sunitinib, pazopanib, temsirolimus, everolimus, axitinib, nivolumab, cabozantinib, lenvatinib mesylate, ipilimumab, pembrolizumab, avelumab

Liver cancer: sorafenib, regorafenib, nivolumab, lenvatinib mesylate, pembrolizumab, cabozantinib, ramuciruma

Lung cancer: bevacizumab, crizotinib, erlotinib, gefitinib, afatinib dimaleate, ceritinib, ramucirumab, nivolumab, pembrolizumab, osimertinib, necitumumab, alectinib, atezolizumab, brigatinib, trametinib, dabrafenib, durvalumab, dacomitinib, lorlatinib, entrectinib

Lymphoma: Ibritumomab tiuxetan, denileukin diftitox, brentuximab vedotin, rituximab, vorinostat, romidepsin, bexarotene, bortezomib, pralatrexate, ibrutinib, siltuximab, idelalisib, belinostat, obinutuzumab, nivolumab, pembrolizumab, rituximab and hyaluronidase human, copanli sib hydrochloride, axicabtagene ciloleucel, acalabrutinib, ti sagenlecleucel, venetoclax, mogamulizumab-kpkc, duvelisib, polatuzumab vedotin-piiq, zanubrutinib

Microsatellite instability-high or mismatch repair-deficient solid tumors: pembrolizumab

Multiple myeloma: bortezomib, carfilzomib, panobinostat, daratumumab, ixazomib citrate, elotuzumab, selinexor

Myelodysplastic/myeloproliferative disorders: imatinib mesylate, ruxolitinib phosphate, fedratinib hydrochloride

Neuroblastoma: dinutuximab

Ovarian epithelial/fallopian tube/primary peritoneal cancers: bevacizumab, olaparib, rucaparib camsylate, niraparib tosylate monohydrate

Pancreatic cancer: erlotinib, everolimus, sunitinib, olaparib

Prostate cancer: cabazitaxel, enzalutamide, abiraterone acetate, radium 223 dichloride, apalutamide, darolutamide

Skin cancer: vismodegib, sonidegib, ipilimumab, vemurafenib, trametinib, dabrafenib, pembrolizumab, nivolumab, cobimetinib, alitretinoin, avelumab, encorafenib, binimetinib, cemiplimab-rwlc

Soft tissue sarcoma: pazopanib, alitretinoin

Solid tumors with an NTRK gene fusion: larotrectinib sulfate, entrectinib

Stomach (gastric) cancer: pembrolizumab, trastuzumab, ramucirumab

Systemic mastocytosis: imatinib mesylate, midostaurin

Thyroid cancer: cabozantinib, vandetanib, sorafenib, lenvatinib mesylate, trametinib, dabrafenib

The drugs described above are either small molecules or antibodies. These molecules and targeted drugs developed in the future can be encapsulated in PLGA microparticles as a combination targeted therapy in the combinatorial targeted therapy method disclosed herein. If necessary, chemotherapy drugs can be also added into the combination therapy.

PLGA Microparticles

As described previously, the above targeted drugs include hormone therapies, signal transduction inhibitors, gene expression modulators, apoptosis inducers, angiogenesis inhibitors, immunotherapies, and toxin delivery molecules. Each drug group treats cancer caused by different genomic alterations. In addition, each drug group has its own subgroups. For example, signal transduction is associated with cell cycle progression and cell growth and related to 10 oncogenic signaling pathways. These 10 oncogenic signaling pathways are receptor tyrosine kinase/MAPK pathway, PI3K pathway, NRF2 pathway, TGFβ pathway, WNT pathway, MYC pathway, TP53 pathway, cell cycle pathway, HIPPO pathway and Notch pathway. Again each pathway has many proteins involved in various cell-related mechanisms. The disclosed method combines targeted drugs which block or inhibit two or more proteins from two or more groups, two or more subgroups, or two or more pathways for treating various cancer types. As an example, the present invention can combine inhibitors of receptor tyrosine kinase/MAPK pathway and PI3K pathway for treating various cancer types. Genomic alterations of these two pathways are associated with more than 60% of cancer patients.

PLGA is a biodegradable polymer with an excellent safety profile. A number of products with a drug encapsulated in PLGA are already approved by FDA. PLGA is a copolymer of lactic acid and glycolic acid. PLGA and a drug can be fabricated into microparticles including microcapsules and microspheres. Microcapsules generally have a drug core coated with a polymer film and may be spherical or non-spherical in shape. In contrast, microspheres have drugs dispersed evenly in polymer and are spherical in shape.

PLGA microparticles are a valuable drug delivery system due to their versatility in controlling drug release rate. The drug release rate from PLGA microparticle can be controlled by adjusting a number of parameters such as 1) ratio between polylactic acid (PLA) and polyglycolic acid (PGA), 2) molecular weight and 3) size of micro-particle.

In PLGA, polylactic acid is more hydrophobic compared to polyglycolic acid and subsequently hydrolyzes (i.e., degrades) slower. For example, PLGA 50:50 (PLA:PGA) exhibits a faster degradation than PLGA 75:25 due to preferential degradation of glycolic acid proportion if two polymers have the same molecular weights. PLGA with higher molecular weight exhibits a slower degradation rate than PLGA with lower molecular weight. Molecular weight has a direct relationship with the polymer chain size. Higher molecular weight PLGA has longer polymer chain and requires more time to degrade than lower molecular weight PLGA. In addition, an increase in molecular weight decreases drug diffusion rate and therefore drug release rate.

The size of a micro-particle also affects the rate of drug release. As the size of a micro-particle decreases, the ratio of surface area to volume of the micro-particle increases. Thus, for a given rate of drug diffusion, the rate of drug release from the micro-particle will increase with decreasing micro-particle size. In addition, water penetration into smaller micro-particle may be quicker due to the shorter distance from the surface to the center of the micro-particle.

In addition, the property and amount of drug can also affect the rate of drug release.

In an example, the drug powder disclosed herein uses microparticles having sizes between 1 μm and 250 μm, preferably less than 50 μm. The composition of PLGA preferably includes a ratio equal to or more than 50% by weight of polylactic acid (PLA). In one preferred embodiment, each PLGA micro-particle contains 1-50% of drug by weight. Molecular weight of PLGA may be between 7,000 and 150,000 Daltons, preferably 30,000 to 150,000 Daltons.

PLGA Microparticle Fabrication

Microparticles in the combinatorial targeted therapy method disclosed herein can be prepared by microencapsulation, spray drying, precipitation, hot melt microencapsulation, co-extrusion, precision particle fabrication (PPF) or other fabrication techniques. Microencapsulation techniques may use single, double or multiple emulsion process in combination with solvent removal step such as evaporation, extraction or coacervation step. They are the most commonly used techniques to prepare micro-particles. The above techniques including the microencapsulation techniques can be used for water soluble drug, organic solvent soluble drug and solid powder drug. The combinatorial targeted therapy method disclosed herein may also use hydrogel overcoating onto the surface of PLGA microparticles to extend the duration of drug release.

Hydrogel

Hydrogel is a hydrophilic polymer that can swell in water and hold a large amount of water. A three-dimensional structure results from the hydrophilic polymer chains held by crosslinks. The hydrogel is a very good absorbent which can absorb a large amount of water up to more than 10 times its own weight. It is used for many applications such as scaffolds in tissue engineering, sustained drug delivery system, breast implant, wound dressing, disposable diaper and other applications. The hydrogel can be prepared from synthetic polymer or natural polymer. The synthetic polymer includes polyhydroxyethyl methacrylate (PHEMA), polyethylene glycol (PEG), polyvinyl alcohol (PVA), polyvinylpyrrolidone (PVP), polyimide (PI), polyacrylate (PA), polyurethane (PU) and other synthetic polymers. The natural polymer includes collagen, hyaluronic acid, alginate, chitosan and other natural polymers.

Again, in one embodiment, the combinatorial targeted therapy method disclosed herein uses hyaluronic acid (HA) as its hydrogel component. It is a linear polysaccharide formed from N-acetyl-D-glucosamine and glucuronic acid with a molecular weight ranging from 2×10⁵ to 1×10⁷ daltons. It is naturally abundant in biological fluids and tissues. It is biocompatible, biodegradable, non-immunogenic and non-toxic. HA is used in many clinical applications such as intra-articular injection for treating osteoarthritis patients, wound healing, treating dry eye and other applications. Again, in an example, the drug powder is made by overcoating PLGA-drug microparticles with hyaluronic acid.

The HA-overcoated PLGA drug microparticles disclosed herein have many advantages over non-coated PLGA-drug microparticles. Some of these advantages are improved immunogenicity, potential zero-order drug release and longer drug release time.

Thus, the combinatorial aspects of the targeted treatment method disclosed herein include selecting and combining several drugs for specific targeting at each cancer site, selecting the appropriate delivery method for each individual drug, and simultaneously delivering each targeted drug to its proper cancer site and through its appropriate delivery mechanism.

Combinatorial targeted therapy provides solutions to the limitations faced by current cancer therapies in order to maximize efficacy, reduce the limitations associated with controlling the resistances of cancer cells to targeted drugs and minimize systemic side effects. The application of the aforementioned rules allows for the proper selection of delivery methods for each targeted therapy at each cancer site. Due to the advantages inherent to local delivery methods, monitoring of tumor size or volume may be followed up with proper adjustments to dosages, further optimizing efficacy and minimizing toxicity. Through the advances brought forth by combinatorial targeted therapy, additional treatments may become available to cancer patients with few treatment options, including advanced stage cancer (metastatic cancer) and pancreatic cancer patients. By overcoming the limitations that exist for current cancer therapies, combinatorial targeted therapy can provide life-saving treatment for those who would otherwise be untreatable.

### EXAMPLES

As mentioned previously, treating cancer with a single targeted drug works initially. However, cancer cells develop resistance to this single targeted drug over time and cancer grows again. In order to reduce the issues that arise from these cancer cells' resistance to the targeted drug, another targeted drug(s) can be added to make a combination targeted therapy. As an example of the combination targeted therapy, sorafenib (MAPK pathway and RAF inhibitor) encapsulated in PLGA and everolimus (PI3K pathway and mTOR inhibitor) encapsulated in PLGA were combined and tested using a mouse model.

Preparation of Sorafenib-PLGA Microspheres

PLGA (1 g) was dissolved in 9.5 mL of dichloromethane (DCM) by stirring at room temperature (RT) for 1 hour (h). To the polymer solution, sorafenib (SOF: 200 mg) dissolved in 0.5 mL of dimethyl sulfoxide (DMSO) was added and stirred for an additional 10 minutes (min). The solution (oil phase) was poured into the dispersion phase tank of SPG membrane machine manufactured by MCTech and pressed through ceramic membrane with a pore size of 20 μm or 30 μm using nitrogen gas into the continuous phase tank filled with 4% polyvinyl alcohol (PVA) solution. This process is being carried out for about 2 h. The aqueous phase was transferred into a glass beaker and stirred with propeller stirrer for 4 h at RT to remove DCM. Then, cold deionized water (DI, 500 mL) was added to the microsphere solution and filtered on 20 μm filter paper or centrifuged at 3,000 rpm for 5 min after cooling down for 6 h at 4° C., followed by washing with cold water (1 L). The collected pellets were freeze-dried for 24-48 h and vacuum dried for 72-96 h at 39° C. in vacuum oven.

Preparation of Everolimus-PLGA Microspheres

PLGA (1 g) was dissolved in 9 mL of dichloromethane (DCM) by stirring at room temperature (RT) for 1 h. To the polymer solution, everolimus (EVE: 100 mg) was added and stirred for additional 10 minutes. The solution (oil phase) was poured into the dispersion phase tank of SPG membrane machine manufactured by MCTech and pressed through ceramic membrane (a pore size of 30 μm) using nitrogen gas into the continuous phase tank filled with 4% PVA solution. Then, the same process described in the above example was followed.

In Vitro Release Study of Sorafenib and Everolimus

The in vitro release study was carried out by sample-and-separate method. Briefly, 5 mg of microsphere sample (n=3) was taken into 100 mL flask and dispersed in 50 mL of release medium (0.5% Tween 20 and 0.1% sodium azide in phosphate-buffered saline (PBS at pH 7.4)). The flasks were placed in orbital agitating incubator at 37° C. and shacked at 100 rpm. At certain time points, 40 mL medium was taken and centrifuged at 3000 rpm for 2 min. From the supernatant, 30 mL was pipetted and replaced by same amount of the fresh media. In the collected supernatant, the content of the released SOF or EVE was analyzed by high performance liquid chromatography (HPLC). Since EVE was known to degrade quickly in aqueous solution as free form, we decided to analyze EVE content in the microsphere residue. We confirmed that EVE was stable inside the microsphere.

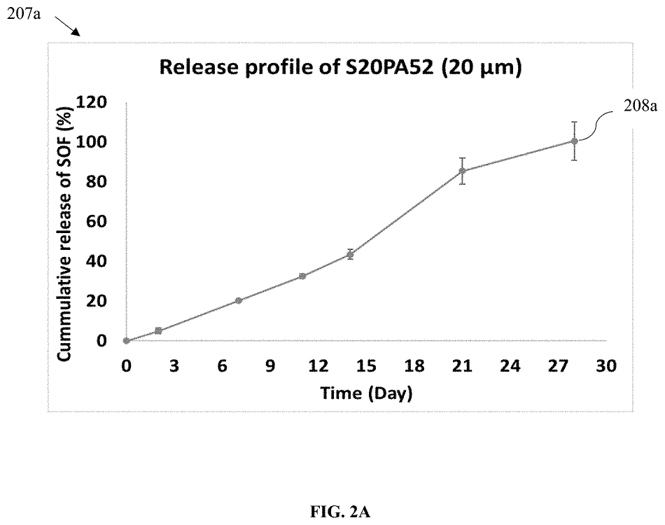

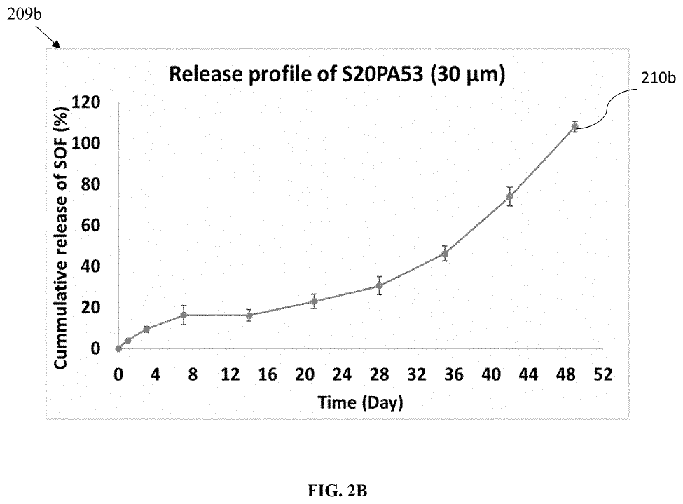

Sorafenib was encapsulated in two different PLGA polymers with acid terminated PLGA with molecular weight (MW) of 20,000 daltons and a ratio of PLA:PGA=50:50 (S20PA52) (207a) and MW of 30,000 daltons and a ratio of PLA:PGA=50:50 (S20PA53) (209b). S20PA52 was prepared using a membrane with a pore size of 20 μm which produced a mean size of 30 μm microspheres while S20PA53 with a pore size of 30 μm which produced a mean size of 47 μm microspheres. The in vitro sorafenib release profiles of these two microspheres are described in FIGS. 2A and 2B, respectively. S20PA53 with larger MW PLGA and larger size of microspheres (100% release in about 47 days) (210b) releases sorafenib much slower than S20PA52 (100% release in about 30 days) (208a).

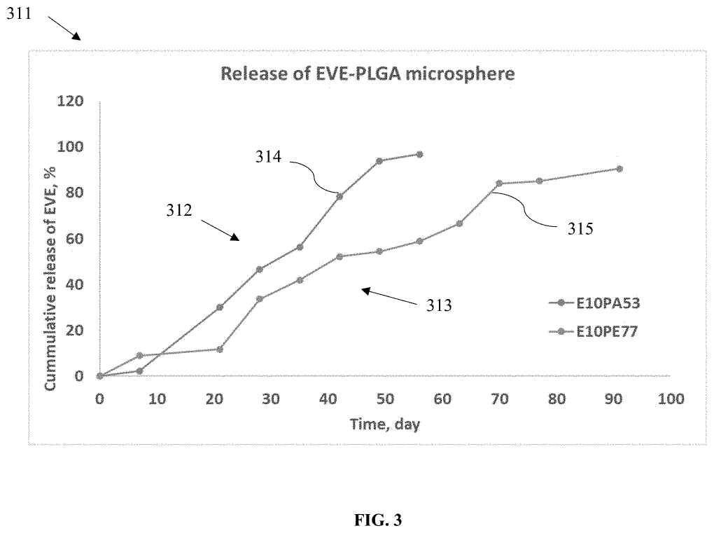

Everolimus was also encapsulated in two different PLGA polymers (311) (acid terminated PLGA with MW of 30,000 daltons and a ratio of PLA:PGA=50:50 (E10PA53) (312) and ester terminated PLGA with MW of 70,000 daltons and a ratio of PLA:PGA=75:25 (E10PE77) (313). The in vitro everolimus release profiles of these two microspheres are described in FIG. 3. E10PE77 with larger MW and higher ratio of PLA:PGA (80% release in 70 days) (315) releases everolimus much slower than E10PA53 (80% release in 40 days) (314).

The above demonstrates that it is possible to encapsulate both sorafenib and everolimus in PLGA polymers, and thus control their release at the cancer site, which is needed when administering them via local delivery, according to the combinatorial targeted cancer treatment method disclosed herein.

It may be advantageous to set forth definitions of certain words and phrases used in this patent document.

The term “combinatorial targeted therapy” refers to the simultaneous delivery at various cancer sites of multiple targeted drugs through both systemic and local means. The term “combination targeted therapy” refers only to the delivery of multiple targeted drugs.

The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims.

If present, use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the aspects, embodiments or examples shown should be considered as exemplars, rather than limitations on the apparatus or procedures disclosed or claimed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

If means-plus-function limitations are recited in the claims, the means are not intended to be limited to the means disclosed in this application for performing the recited function, but are intended to cover in scope any equivalent means, known now or later developed, for performing the recited function.

If any presented, the claims directed to a method and/or process should not be limited to the performance of their steps in the order written, and one skilled in the art can readily appreciate that the sequences may be varied and still remain within the spirit and scope of the present invention.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples. Hence, the scope of the invention is defined by the accompanying claims and their equivalents. Further, each and every claim is incorporated as further disclosure into the specification.

1. A combinatorial targeted therapy method for treating cancer, the method comprising:
performing initial molecular diagnostics to detect genomic alterations at each cancer site of a subject;
for each cancer site, designing an initial combination targeted therapy by selecting a plurality of targeted drugs, based on the results of the initial molecular diagnostic at each cancer site;
assigning each targeted drug to systemic or local delivery method, based on each targeted drug's properties;
simultaneously treating all cancer sites according to the designed initial combination targeted therapy for each site, by delivering each targeted drug according to assigned delivery method to each targeted drug;
monitoring the progress of the cancer at each cancer site by performing follow-up molecular diagnostics at each cancer site; and
for each cancer site, maintaining the initial combination targeted therapy or designing a follow-up combination targeted therapy, based on the results of the follow-up molecular diagnostics at each cancer site.

2. The method of claim 1 wherein the systemic delivery comprises oral and IV drug delivery.

3. The method of claim 1 wherein drug's properties include known systemic toxicity of the drug and potency of the drug.

4. The method of claim 1 wherein the assigning of each targeted drug to systemic or local delivery is further based on the location of each cancer site.

5. The method of claim 3, wherein each high potency targeted drug, requiring less than 500 mg oral daily dose, is assigned to local delivery.

6. The method of claim 1 wherein each targeted drug that is assigned to local delivery is encapsulated in a biodegradable polymer for sustained and controlled release of the targeted drug at the cancer site.

7. The method of claim 6, wherein the biodegradable polymer is PLGA.

8. The method of claim 1, wherein one targeted drug is in the form of microparticles made of sorafenib or other MAPK pathway inhibitor and PLGA, for sustained and controlled release of the targeted drug at the cancer site.

9. The method of claim 1, wherein one targeted drug is in the form of microparticles made of everolimus or other PI3K pathway inhibitor and PLGA, for sustained and controlled release of the targeted drug at the cancer site.

10. A combinatorial targeted therapy method for treating metastatic cancer in a subject, the method comprising preventing unacceptable level of systemic toxicity in the subject by:
performing initial molecular diagnostics to detect genomic alterations at each cancer site of the subject;
for each cancer site, designing an initial combination targeted therapy by selecting a plurality of targeted drugs, based on the results of the initial molecular diagnostic at each cancer site;
assigning each targeted drug to systemic or local delivery method, based on each targeted drug's properties;
simultaneously treating all cancer sites according to the designed initial combination targeted therapy for each site, by delivering each targeted drug according to assigned delivery method to each targeted drug; and
monitoring the progress of the cancer at each cancer site by performing follow-up molecular diagnostics at each cancer site.

11. The method of claim 10, wherein one targeted drug is in the form of microparticles made of sorafenib or other MAPK pathway inhibitor and PLGA, for sustained and controlled release of the targeted drug at the cancer site.

12. The method of claim 10, wherein one targeted drug is in the form of microparticles made of everolimus or other PI3K pathway inhibitor and PLGA, for sustained and controlled release of the targeted drug at the cancer site.

13. The method of claim 10 wherein drug's properties include known systemic toxicity of the drug and potency of the drug.

14. The method of claim 10 wherein the assigning of each targeted drug to systemic or local delivery is further based on the location of each cancer site.

15. The method of claim 10, wherein each high potency targeted drug, requiring less than 500 mg oral daily dose, is assigned to local delivery.
